(19) United States
(12) Patent Application Publication
(10) Pub. No.: US 2007/0156070 A1
(43) Pub. Date: Jul. 5, 2007

(54) IV Compatible limb stabilization apparatus

(76) Inventor: Lester M. Schwab, Alamo, CA (US)

Correspondence Address: LARRY D. JOHNSON, P.O. BOX 470277, CELEBRATION, FL, 34747, US

(21) Appl. No.: 10/586,999

(22) PCT Filed: Jan. 21, 2005
(86) PCT No.: PCT/US05/01942

§ 371 Date: Jul. 20, 2006

Related U.S. Application Data
(60) Provisional application No. 60/537,670, filed on Jan. 20, 2004.
(60) Provisional application No. 60/578,190, filed on Jun. 9, 2004.

(51) Int. Cl. A61F 5/00

(52) U.S. Cl. 602/5

(57) ABSTRACT

A splint apparatus for use with intravenous therapy includes an upper portion adapted to be positioned above a patient's limb joint, and a lower portion connected to the upper portion at an angle from 0 to 90 degrees, and preferably between 30 and 60 degrees, the lower portion including a pair of sides and a central aperture for passage of an intravenous catheter. The lower portion is adapted to be positioned and secured below a patient's limb joint, so that the upper portion prevents flexion of the patient's limb joint beyond the defined angle. The apparatus provides an inexpensive method of limb restraint allowing free movement of the limb with the ability for the patient to stand, sit, or lay down easily and without discomfort, while simultaneously preventing bending the limb more than a predetermined angle so as to allow a free flow of IV fluids.

### BACKGROUND OF THE INVENTION

1. Technical Field

The present invention relates generally to veterinary and medical implements, and more particularly to an improved limb stabilization apparatus or splint.

2. Background Art

Medical conditions in companion animals may require the placement of intravenous catheters and fluid therapy. In most instances the cephalic vein in the foreleg of the animal is used. Once the catheter is in place the hub of the needle is taped, and then that tape is wound around the leg and the male end of the primary IV set is joined to the catheter hub. Several lengths of adhesive tape are then used to secure the catheter and IV set. The end result is the inability of the doctor to view this union, and the probability that the IV catheter will inadvertently be dislodged when removing the tape to flush the catheter or change IV solutions.

Such catheters have been placed in companion animals in the same manner for over sixty years, and during this time various methods of leg restraint have been attempted but none have worked well. Present methods of leg restraint place plastic splints ventrally on the leg, which, if attached both below and above the elbow severely restrain the animals ability to move about and are very uncomfortable. Only one known prior art splint (U.S. Pat. No. 4,440,159 to Cochran) does not use the ventral approach, but this method is extremely uncomfortable and does not work properly as the splint slips off of the shoulder.

Three basic problems exist. First, animals will attempt to chew out or pull out the IV catheter necessitating that it be replaced in the opposite leg causing additional discomfort to the patient and increased cost to the doctors and pet owner.

Next, if the animal lays down and/or bends the leg more than 45 degrees it will slow down or stop the IV fluid flow causing an insufficient amount of fluid to be delivered. If the flow stops a clot may form, necessitating flushing the line out or replacing in the other leg again, causing increased discomfort to the patient and additional cost to the doctors and to the owner. It is not uncommon to inadvertently pull out the catheter while attempting to flush the catheter.

Finally, most restraints attach ventrally and may immobilize the elbow. Such restraints make it difficult for the animal to stand and extremely uncomfortable to sit or lay down. If the animal attempts to lie down it must do so by stretching the leg straight out from the shoulder area.

IV therapy in humans poses similar problems. For example, IV infusion equipment may be used in conjunction with a padded board bound to the arm ventrally and extending from the wrist to above the elbow. This is extremely uncomfortable, and the patient suffers from a very stiff arm.

In IV therapy, whether on an intravenous infusion pump or not, a specific measured amount of fluid and/or drugs are predetermined to be given during a specific time frame. This delivery system may be interrupted by the patient's flexion of their arm. This can result in clot formation requiring the catheter to be replaced, or triggering of a pump alarm necessitating staff intervention.

The foregoing patents and discussion reflect the current state of the art of which the present inventor is aware. Reference to, and discussion of, this information is intended to aid in discharging Applicant's acknowledged duty of candor in disclosing information that may be relevant to the examination of claims to the present invention. However, it is respectfully submitted that none of the above-indicated patents or information disclose, teach, suggest, show, or otherwise render obvious, either singly or when considered in combination, the invention described and claimed herein.

### DISCLOSURE OF INVENTION

The IV compatible limb stabilization apparatus of the present invention provides a splint apparatus for use with intravenous therapy. The splint apparatus includes an upper portion adapted to be positioned above a patient's limb joint, and a lower portion connected to the upper portion at an angle from 0 to 90 degrees, and preferably between 30 and 60 degrees, the lower portion including a pair of sides and a central aperture for passage of an intravenous catheter. The lower portion is adapted to be positioned and secured below a patient's limb joint, so that the upper portion prevents flexion of the patient's limb joint beyond the defined angle.

The present invention thus provides an improved limb stabilization apparatus or splint for use with intravenous therapy, providing fast and easy limb stabilization and access to the union of an IV catheter and primary IV set. The inventive apparatus may be used with animals, but has particular human application, as for use with pediatric medicine, sedated patients, patients requiring long term IV therapy, and patients in restraint.

A principal object of the invention is to provide an inexpensive method of limb restraint allowing free movement of the limb with the ability for the patient to stand, sit, or lay down easily and without discomfort, while simultaneously preventing bending the limb more than a predetermined angle so as to allow a free flow of IV fluids.

This invention allows for decreasing the amount of taping required to secure the union of the IV catheter and primary IV set. The less tape used the less likely it would be to inadvertently dislodge the IV catheter while reaching this union to flush the IV catheter.

The present invention provides a novel splint apparatus which is easily placed dorsally on the foreleg of an animal with minimal discomfort, as there is no weight bearing and no restraint above the elbow. The animal may move about freely, lay or stand or sit but cannot flex the leg more than a predetermined angle, allowing for the IV solutions to flow freely.

In human application, use of the inventive stabilizer prevents over flexion of the aim which may slow IV fluid and trigger an infusion pump alarm, while allowing for a measure of freedom of arm movement.

It is therefore an object of the present invention to provide a new and improved limb stabilization apparatus or splint for IV therapy.

It is another object of the present invention to provide a new and improved limb stabilization apparatus that is fast and easy to apply and allows for quick and easy access to the union of the IV catheter and primary IV set.

A further object or feature of the present invention is a new and improved splint that permits comfortable movement by the patient.

An even further object of the present invention is to provide a novel splint apparatus that ensures a free flow of IV fluids.

Other novel features which are characteristic of the invention, as to organization and method of operation, together with further objects and advantages thereof will be better understood from the following description considered in connection with the accompanying drawing, in which preferred embodiments of the invention are illustrated by way of example. It is to be expressly understood, however, that the drawing is for illustration and description only and is not intended as a definition of the limits of the invention. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the national patent office(s) and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of a limb stabilization apparatus of this invention in place on a patient's arm;

FIG. 2 is a perspective view of a limb stabilization apparatus of this invention illustrating optional protective ridges and incremental measurement marks; and

FIG. 3 is a perspective view of one side of the lower portion of a limb stabilization apparatus illustrating optional IV tubing guides.

### BEST MODE FOR CARRYING OUT THE INVENTION

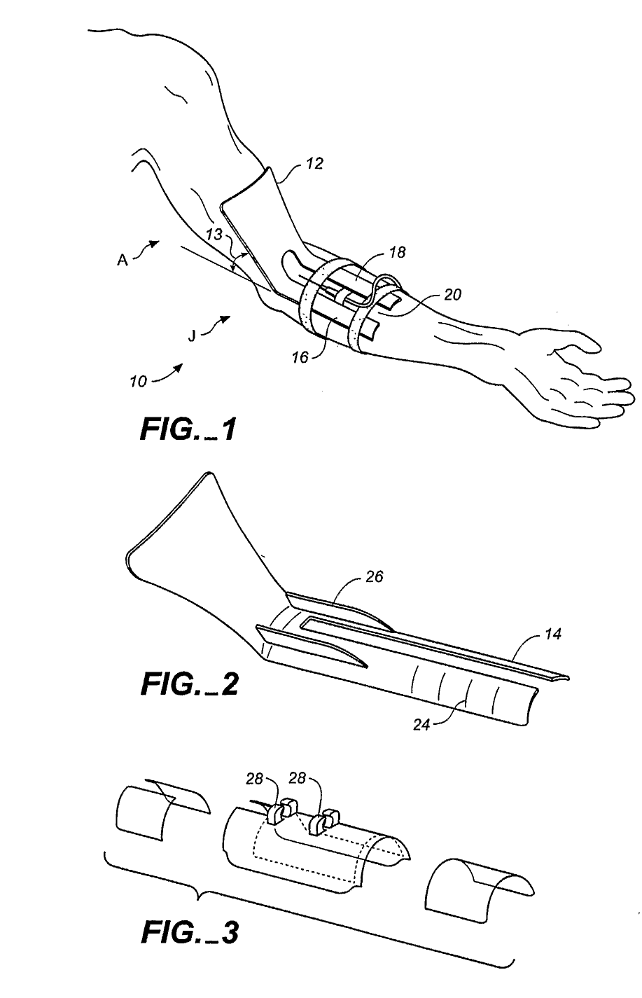

Referring to FIGS. 1 through 3, wherein like reference numerals refer to like components in the various views, FIG. 1 is a perspective view of a limb stabilization apparatus 10 in place on a patient's arm A. The splint apparatus 10 includes an upper portion 12 adapted to be positioned above a patient's limb joint J, and a lower portion 14 connected to the upper portion 12 at an angle 13 from 0 to 90 degrees, and preferably between 30 and 60 degrees. The lower portion 14 includes a pair of sides 16, 18 and a central aperture 20 for passage of an intravenous catheter 22. The lower portion 14 is adapted to be positioned and secured below the patient's limb joint, so that the upper portion 12 prevents flexion of the patient's limb joint beyond the angle 13.

FIG. 2 illustrates that the lower portion 14 of the apparatus could be marked with indicia 24 at intervals (e.g., ½ inch) so that doctors or tech may cut the splint to the proper size for the patient. In addition, the medial sides of the lower portion of the splint could include a small ridge 26 (e.g., ⅛ inch) to assist in preventing animal “chew out.”

FIG. 3 illustrates that one arm of the stabilizer may include guides 28 into which the primary IV set tubing can be snapped so as to prevent tangling and twisting of the line, and to decrease tension on the junction of IV catheter and primary IV set.

The splint is preferably made of plastic with an angle of approximately 45 degrees at the elbow portion and arms ventral to that angle that are marked at intervals allowing the doctor or tech to cut the length to suit the majority of patients. Three sizes will accommodate the majority of dogs and cats. Above the elbow the device widens so as to capture the shoulder when the patient attempts to move the arm more than 45 degrees. Only that portion below the elbow is taped and that with a wide tape that sticks only to itself so as to make it easier to reach the union of IV catheter and primary IV set to change solutions or flush out.

The plastic arms of the splinting device that straddle the IV catheter and the wide non-stick tape protects the union of IV catheter and primary IV set, provides a barrier to chewing or pulling out of catheter, and prevents excess bending of the foreleg which allows for a free flow of IV fluids. Above and below the elbow the splints have concavity to shape to the limb.

In veterinary medicine the inventive device is preferably placed dorsally on the leg. In human medicine the device can be placed dorsally or ventrally, but will work best when placed at the crook in the elbow (ventrally).

The units are preferably closed at the bottom and when applied to the arm a gauze pad would be placed at the bottom to prevent chafing of the skin. Only one or two areas ventral to the elbow need to be taped to secure the device to the arm. No taping is required dorsal to the elbow, as the intent is simply to prevent the arm from bending more than 45 degrees.

In veterinary medicine the device should stretch the full length of the leg, and the angle at the elbow is between 30 to 60 degrees, and preferably approximately 47 degrees. In human medicine the device need only sufficient length to properly bind the arm (e.g., three inches), and the angle at the elbow is between 30 to 60 degrees, and preferably approximately 53 degrees. In addition, the notch where the splint arms come together may be extended by one-half inch to accommodate catheter placement.

If complete immobilization of the arm is desired the inventive stabilizer can be manufactured without the cantilever back so as to eliminate the uncomfortable “over the elbow” feeling when using an arm board.

If the catheter is placed in the wrist area, or anywhere on the front of the leg, the stabilizer may be placed in the front of the leg. If the catheter is placed on the back of the leg (in the crook of the elbow) the stabilizer can be placed on the back of the leg. Regardless of where the IV catheter is placed the stabilizer can be placed so that it will accomplish the purpose of limiting the aim flexion and allowing for a free flow of IV fluids.

The inventive splints can be made any length, width, concavity, or angle, and be used regardless of where the IV catheter may be placed. They can be constructed of plastic and be washed or gas sterilized between uses, or they could be constructed of stainless steel and autoclaved.

The foregoing disclosure is sufficient to enable one having skill in the art to practice the invention without undue experimentation, and provides the best mode of practicing the invention presently contemplated by the inventor. While there is provided herein a full and complete disclosure of the preferred embodiments of this invention, it is not intended to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Accordingly, the proper scope of the present invention should be determined only by the broadest interpretation of the appended claims so as to encompass all such modifications as well as all relationships equivalent to those illustrated in the drawings and described in the specification.

What is claimed as invention is:

1. A splint apparatus for use with intravenous therapy, said splint apparatus comprising:
an upper portion adapted to be positioned above a patient's limb joint; and
a lower portion connected to said upper portion at an angle from 0 to 90 degrees, said lower portion including a pair of sides and a central aperture for passage of an intravenous catheter, said lower portion adapted to be positioned below a patient's limb joint, wherein when said lower portion is secured below a patient's limb joint, said upper portion prevents flexion of the patient's limb joint beyond said angle.

2. The splint apparatus of claim 1 wherein said angle is between 30 and 60 degrees.

3. The splint apparatus of claim 1 for use in veterinary medicine, and wherein said angle is approximately 47 degrees.

4. The splint apparatus of claim 1 for use in human medicine, and wherein said angle is approximately 53 degrees.

5. The splint apparatus of claim 1 wherein said lower portion includes incremental measurement marks to properly size the apparatus to the patient.

6. The splint apparatus of claim 1 wherein said lower portion includes raised ridges to limit access to said aperture.

7. The splint apparatus of claim 1 wherein at least one side of said lower portion includes guides for capture of intravenous tubing.
